(19) United States
(12) Patent Application Publication
(10) Pub. No.: US 2025/0281218 A1
(43) Pub. Date: Sep. 11, 2025

(54) CATHETER ACTUATOR WITH 360 DEGREE ACCESSIBILITY AND INTEGRATED LOCK

(71) Applicant: Boston Scientific Scimed, Inc., Maple Grove, MN (US)

(72) Inventors: Dustin J. Olynyk, Eagan, MN (US); James Holmberg, Champlin, MN (US); Garth Mindermann, III, Bloomington, MN (US); Michael Strawn, Minneapolis, MN (US)

(21) Appl. No.: 19/074,099

(22) Filed: Mar. 7, 2025

Related U.S. Application Data
(60) Provisional application No. 63/563,089, filed on Mar. 8, 2024.

(51) Int. Cl. A61B 18/00

(52) U.S. Cl. CPC A61B 18/00; A61B 2018/00172; A61B 2018/00184; A61B 2018/00267; A61B 2018/00946

(57) ABSTRACT

A medical catheter comprising a tubular outer shaft, an inner shaft slidably disposed within the outer shaft and dimensioned to extend distally beyond the outer shaft, a radially expandable and radially collapsible spline assembly comprising a plurality of splines, a handle coupled to the proximal end of the outer shaft, wherein the proximal portion of the inner shaft is slidably disposed within the handle, an actuator comprising a slider knob slidably disposed about and fully circumscribing the outer surface of the handle body, wherein the slider knob is operatively coupled to the proximal portion of the inner shaft such that axial displacement of the slider knob by the user causes the inner shaft to translate axially relative to the outer shaft to selectively control a configuration of the spline assembly.

### CROSS REFERENCE TO RELATED APPLICATIONS

The present application claims priority to U.S. Provisional Patent Application No. 63/563,089, filed Mar. 8, 2024, the entire disclosure of which is incorporated herein by reference.

### TECHNICAL FIELD

The present disclosure relates to medical systems and methods for cardiac ablation, and more particularly, to ablation catheters for use in ablating cardiac tissues.

### BACKGROUND

Ablation procedures (e.g., pulsed field ablation, radiofrequency ablation, cryoablation, and the like) are used to treat many different conditions in patients. Ablation can be used to treat cardiac arrhythmias, benign tumors, cancerous tumors, and to control bleeding during surgery. There is a continuing need for ablation catheters with features that improve usability and clinical efficiency.

### SUMMARY

In Example 1, a medical catheter comprising a tubular outer shaft, an inner shaft, a radially expandable and radially collapsible spline assembly, a handle and an actuator. inner shaft is slidably disposed within the outer shaft and is dimensioned to extend distally beyond the outer shaft. The spline assembly comprises a plurality of splines each having a spline proximal end attached to the distal end of the outer shaft, and an opposite spline distal end attached to the distal end of the inner shaft. The handle has a handle proximal end, and a handle distal end, wherein the proximal end of the outer shaft is attached to and extends from the handle proximal end, and a proximal portion of the inner shaft is slidably disposed within the handle. The actuator comprises a slider knob slidably disposed about and fully circumscribing the outer surface of the handle, wherein the slider knob is operatively coupled to the proximal portion of the inner shaft such that axial displacement of the slider knob by a user causes the inner shaft to translate axially relative to the outer shaft to selectively control a configuration of the spline assembly.

In Example 2, the medical catheter of Example 1, wherein the actuator further comprises an inner actuator member attached to and extending from the slider knob into the handle, and wherein the inner actuator member is fixedly secured to the proximal portion of the inner shaft within the handle and operatively couples the inner shaft to the slider knob.

In Example 3, the medical catheter of Example 2, wherein the inner actuator member includes a hub disposed about and fixedly secured to the proximal portion of the inner shaft, and a radial extension extending from the hub to the slider knob.

In Example 4, the medical catheter of Example 3, wherein the handle includes an axial slot defined by opposed first and second lateral edges, and wherein the radial extension extends through the axial slot.

In Example 5, the medical catheter of Example 4, wherein the first and second lateral edges constrain rotation of the inner actuator member during axial displacement of the slider knob.

In Example 6, the medical catheter of any of Examples 1-5, wherein the actuator is configured such that the user can lock the slider knob in an axial position.

In Example 7, the medical catheter of any of Examples 1-6, wherein the slider knob includes an outer knob element that is rotatable about the handle, wherein rotation of the outer knob element by the user in a first direction locks the axial position of the slider knob, and rotation of the outer knob element in a second direction opposite the first direction permits the slider knob to be axially displaced.

In Example 8, the medical catheter of Example 7, wherein the slider knob further comprises an inner knob element that is rotationally fixed relative to the handle and includes a forward portion disposed radially inward of and in threaded engagement with the outer knob element, wherein rotation of the outer knob element causes the inner knob element to translate axially to selectively lock and unlock the slider knob relative to the handle.

In Example 9, the medical catheter of Example 8, wherein the outer knob element includes a forward inner surface, and wherein the handle includes a compression element disposed about the handle between the forward inner surface and the forward portion of the inner knob element, wherein rotation of the outer knob element in the first direction causes the inner knob element to move in a distal direction to compress the compression element between the handle, the forward inner surface of the outer knob element, and the forward portion of the inner knob element to lock the axial position of the slider knob.

In Example 10, the medical catheter of Example 9, wherein the compression element is an O-ring.

In Example 11, the medical catheter of any of Examples 1-5, wherein the actuator further comprises a locking arrangement configured to releasably lock the actuator in one or more pre-defined axial positions.

In Example 12, the medical catheter of Example 11, wherein the first lateral edge includes a first detent at a first location along the lateral edge, and wherein the locking arrangement comprises a locking pin extending radially from the hub through the axial slot, wherein the locking pin is biased toward the first lateral edge so as to automatically engage the first detent to releasably lock the axial position of the slider knob corresponding to the first location.

In Example 13, the medical catheter of Example 12, wherein the locking arrangement further includes a torsion spring disposed about the hub, the torsion spring including a first free end frictionally engaged with an inner surface of the handle, and an opposite second free end positioned to engage and apply a biasing force to the locking pin to bias the locking pin toward the first lateral edge.

In Example 14, the medical catheter of Example 13, wherein the slider knob is configured to be rotatable to counteract the biasing force and selectively disengage the locking pin from the first detent.

In Example 15, the medical catheter of Example 14, wherein the first lateral edge includes a second detent at a second location along the first lateral edge, and a third detent at a third location along the first lateral edge, the first, second and third locations being spaced from one another and corresponding to pre-determined configurations of the spline assembly.

In Example 16, a medical catheter comprising a catheter body, a spline assembly, a handle and an actuator. The catheter body comprises a tubular outer shaft having a proximal end and an open distal end opposite the proximal end, and an inner shaft slidably disposed within the outer shaft having a proximal portion and an opposite distal end, the inner shaft being dimensioned such that the inner shaft distal end extends distally beyond the distal end of the outer shaft. The spline assembly comprises a plurality of splines each having a spline proximal end attached to the distal end of the outer shaft, and an opposite spline distal end attached to the distal end of the inner shaft, the spline assembly configured to be transitioned between a collapsed configuration dimensioned for slidable insertion through a delivery sheath, and an expanded configuration. The handle comprises a handle body having a body proximal end, a body distal end, an outer surface extending from the body proximal end to the body distal end, the handle body defining an interior chamber, wherein the proximal end of the outer shaft is attached to and extends from the body proximal end, and the proximal portion of the inner shaft is disposed within the interior chamber. The actuator is slidably coupled to the handle body between the body proximal end and the body distal end, the actuator comprising a slider knob disposed about and fully circumscribing the outer surface of the handle body such that the slider knob can be axially displaced by a user, wherein the slider knob is further operatively coupled to the proximal portion of the inner shaft such that axial displacement of the slider knob by the user causes the inner shaft to translate axially relative to the outer shaft to transition the spline assembly between the collapsed configuration and the expanded configuration.

In Example 17, the medical catheter of Example 16, wherein the actuator further comprises an inner actuator member attached to and extending from the slider knob into the interior chamber of the handle, and wherein the inner actuator member is fixedly secured to the proximal portion of the inner shaft within the interior chamber and operatively couples the inner shaft to the slider knob.

In Example 18, the medical catheter of Example 17, wherein the inner actuator member includes a hub disposed about and fixedly secured to the proximal portion of the inner shaft, and a radial extension extending from the hub to the slider knob.

In Example 19, the medical catheter of Example 18, wherein the handle body includes an axial slot defined by opposed first and second lateral edges, and wherein the radial extension extends through the axial slot and the first and second lateral edges constrain rotation of the inner actuator member during axial displacement of the slider knob.

In Example 20, the medical catheter of Example 19, wherein the actuator is configured such that the user can lock the slider knob in an axial position.

In Example 21, the medical catheter of Example 20, wherein the slider knob includes an outer knob element that is rotatable about the handle body, wherein rotation of the outer knob element by the user in a first direction locks the axial position of the slider knob, and rotation of the outer knob element in a second direction opposite the first direction permits the slider knob to be axially displaced.

In Example 22, the medical catheter of Example 21, wherein the slider knob further comprises an inner knob element that is rotationally fixed relative to the handle body and includes a forward portion disposed radially inward of and in threaded engagement with the outer knob element, wherein rotation of the outer knob element causes the inner knob element to translate axially to selectively lock and unlock the slider knob relative to the handle body.

In Example 23, the medical catheter of Example 22, wherein the outer knob element includes a forward inner surface, and wherein the handle includes a compression element disposed about the handle body between the forward inner surface and the forward portion of the inner knob element, wherein rotation of the outer knob element in the first direction causes the inner knob element to move in a distal direction to compress the compression element between the handle body outer surface, the forward inner surface of the outer knob element, and the forward portion of the inner knob element to lock the axial position of the slider knob.

In Example 24, the medical catheter of Example 19, wherein the first lateral edge includes a first detent at a first location along the lateral edge, and wherein the locking arrangement comprises a locking pin extending radially from the hub through the axial slot, wherein the locking pin is biased toward the first lateral edge so as to automatically engage the first detent to releasably lock the axial position of the slider knob corresponding to the first location.

In Example 25, the medical catheter of Example 24, wherein the locking arrangement further includes a torsion spring disposed about the hub, the torsion spring including a first free end frictionally engaged with an inner surface of the handle body, and an opposite second free end positioned to engage and apply a biasing force to the locking pin to bias the locking pin toward the first lateral edge.

In Example 26, the medical catheter of Example 25, wherein the slider knob is configured to be rotatable to counteract the biasing force and selectively disengage the locking pin from the first detent.

In Example 27, the medical catheter of Example 26, wherein the first lateral edge includes a second detent at a second location along the first lateral edge, and a third detent at a third location along the first lateral edge, the first, second and third locations being spaced from one another and corresponding to pre-determined configurations of the spline assembly.

In Example 28, a medical catheter comprising a catheter body, a radially expandable and radially collapsible spline assembly, a handle and an actuator. The catheter body comprises a tubular outer shaft and an inner shaft slidably disposed within the outer shaft and dimensioned to extend distally beyond the outer shaft. The spline assembly comprises a plurality of splines each having a spline proximal end attached to the distal end of the outer shaft, and an opposite spline distal end attached to the distal end of the inner shaft. The handle is coupled to the catheter body, the handle having a handle proximal end and a handle distal end, wherein a proximal end of the outer shaft is attached to and extends from the handle proximal end, and a proximal portion of the inner shaft is slidably disposed within the handle. The actuator comprises a slider knob slidably disposed about and fully circumscribing an outer surface of the handle, wherein the slider knob is operatively coupled to the proximal portion of the inner shaft such that axial displacement of the slider knob by a user causes the inner shaft to translate axially relative to the outer shaft to selectively control an amount of expansion of the spline assembly.

In Example 29, the medical catheter of Example 28, wherein the actuator is configured such that the user can lock the slider knob in an axial position.

In Example 30, the medical catheter of Example 28, wherein the actuator further comprises a locking arrangement configured to releasably lock the actuator in one or more pre-defined axial positions.

In Example 31, a handle assembly for a medical catheter having a catheter body including a tubular outer shaft, an inner shaft slidably disposed within the outer shaft, and a radially expandable and collapsible splined electrode assembly disposed at a distal end of the catheter body. The handle assembly comprises a handle and an actuator. The handle comprises a handle body having a body proximal end, a body distal end, an outer surface extending from the body proximal end to the body distal end, the handle body defining an interior chamber configured to slidably receive a proximal portion of the inner shaft. The actuator is slidably coupled to the handle body between the body proximal end and the body distal end, the actuator comprising a slider knob disposed about and fully circumscribing the outer surface of the handle body such that the slider knob can be axially displaced by a user, wherein the slider knob is further configured to be operatively coupled to the proximal portion of the inner shaft such that axial displacement of the slider knob by the user can cause the inner shaft to translate axially relative to the outer shaft to transition the spline assembly between a collapsed configuration and an expanded configuration.

In Example 32, the handle assembly of Example 31, wherein the handle body includes an axial slot defined by opposed first and second lateral edges, and the actuator further comprises an inner actuator member including a hub configured to be disposed about and fixedly secured to the proximal portion of the inner shaft, and a radial extension extending from the hub through the axial slot to the slider knob.

In Example 33, the handle assembly of Example 32, wherein the slider knob includes an outer knob element that is rotatable about the handle body, an inner knob element that is rotationally fixed relative to the handle body and includes a forward portion disposed radially inward of and in threaded engagement with the outer knob element, wherein rotation of the outer knob element causes the inner knob element to translate axially to selectively lock and unlock the slider knob relative to the handle body.

In Example 34, the handle assembly of Example 33, wherein the outer knob element includes a forward inner surface, and wherein the handle further includes a compression element disposed about the handle body between the forward inner surface and the forward portion of the inner knob element, wherein rotation of the outer knob element in a first direction causes the inner knob element to move in a distal direction to compress the compression element between the handle body outer surface, the forward inner surface of the outer knob element, and the forward portion of the inner knob element to lock the axial position of the slider knob.

In Example 35, the handle assembly of Example 32, wherein the first lateral edge includes a plurality of axially-spaced detents, and wherein the actuator includes a locking arrangement comprises a locking pin extending radially from the hub through the axial slot, wherein the locking pin is biased toward the first lateral edge so as to automatically engage the one of the plurality of detents to releasably lock the axial position of the slider knob corresponding to a predetermined configuration of the spline assembly.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1A is a plan view of an exemplary ablation catheter having a splined ablation electrode assembly according to embodiments of the present disclosure.

FIG. 1B is a plan view of the distal end portion of the ablation catheter of FIG. 1A, showing details of the splined ablation electrode assembly, according to embodiments of the present disclosure.

FIGS. 1C-1D are plan views of the distal end portion of the ablation catheter of FIG. 1A showing the spline ablation electrode assembly in various states of deployment.

FIGS. 2A and 2B are partial isometric and cross-sectional views, respectively, of a handle and actuator assembly for an ablation catheter, such as the ablation catheter of FIG. 1A, according to embodiments of the present disclosure.

FIG. 3 is a partial cross-sectional view of an alternative handle and actuator assembly for an ablation catheter, such as the ablation catheter of FIG. 1A, according to embodiments of the present disclosure.

FIGS. 4A-4C are partial isometric views of an alternative handle and actuator assembly for an ablation catheter, such as the ablation catheter of FIG. 1A, according to embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

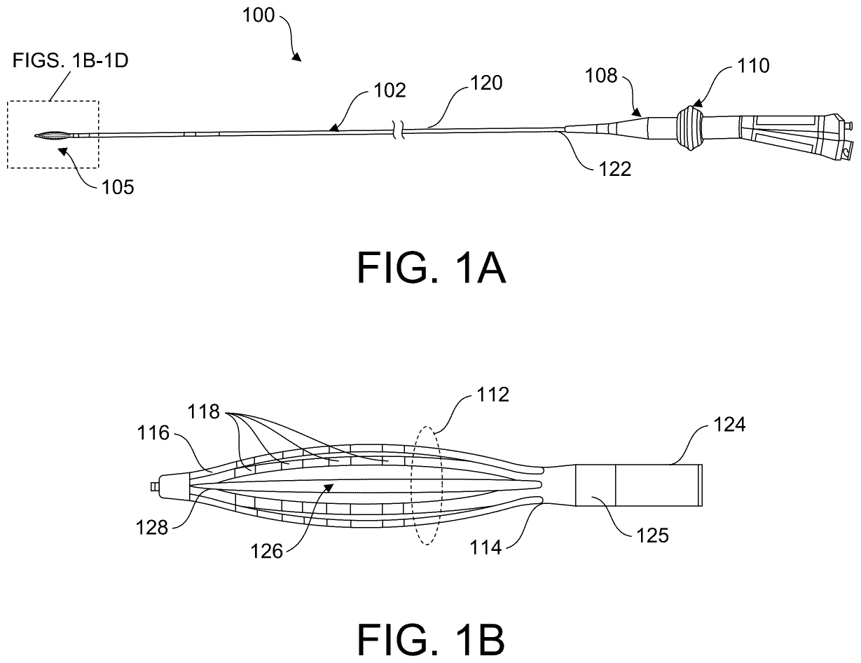

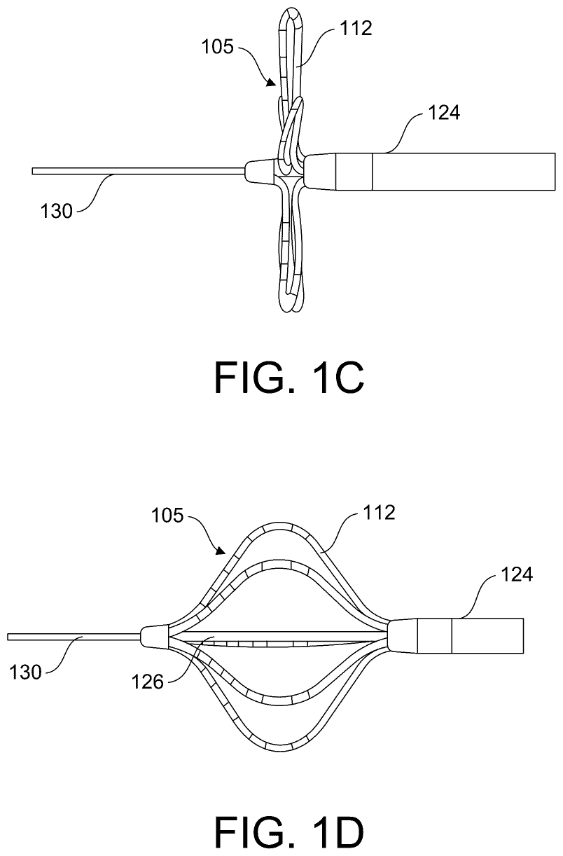

FIG. 1A is a plan view of an exemplary ablation catheter 100 having a splined assembly 105 according to embodiments of the present disclosure. FIG. 1B is a plan view of the distal end portion of the ablation catheter 100, showing, among other things, details of the spline assembly 105, according to embodiments of the present disclosure. In various embodiments, the ablation catheter may be particularly useful in ablation procedures targeting the pulmonary vein ostium of a patient for treating atrial fibrillation. In particular, the ablation catheter 100 may be configured as a pulsed field ablation catheter with electrodes configured to receive pulsed electrical signals/waveforms from a pulse generator, thereby creating pulsed electric fields sufficient for ablating target tissue via irreversible electroporation.

Referring collectively to FIGS. 1A and 1B, as shown, the ablation catheter 100 includes a catheter body 102, the spline assembly 105, a handle 108 and an actuator 110. As further shown, the catheter body 102 extends from the handle 108, and the spline assembly 105 is attached to and extends distally from the catheter body 102. Additionally, the actuator 110 is coupled to the handle 108. As will be explained in greater detail herein, the actuator 110 is movable (i.e., slidable) relative to the handle 108 under action by a clinician and is operable to change the geometric configuration of the spline assembly 105 during use. Additionally, the configuration of the various embodiments of the actuator 110 of the present disclosure facilitate improved ease of use by the clinician, in that the actuator 110 is readily accessible by the user regardless of the orientation of the handle 108.

With particular reference to FIG. 1B, in the illustrated embodiment, the spline assembly 105 includes a plurality of splines 112 each having a spline proximal end 114 and an opposite spline distal end 116, with each spline carrying a plurality of electrodes 118. For clarity of illustration, only a single spline 112 is labeled in FIG. 1B, although the skilled artisan will readily recognize that the referenced features apply to all of the illustrated splines. The skilled artisan will also readily recognize that the particular number of splines 112 and electrodes 118 illustrated in FIG. 1B is merely exemplary, and thus embodiments with more or fewer splines 112 or electrodes 118 fall within the scope of the present disclosure. Additionally, the size and shape of the illustrated electrodes 118 is similarly exemplary. In embodiments, the electrodes 118 may be operable as ablation electrodes, sensing electrodes, or tracking electrodes, e.g., as used for impedance tracking of the location of the spline assembly 105 in electroanatomical mapping systems, or combinations of the foregoing.

As further shown, the catheter body 102 includes an outer shaft 120 having a proximal end 122, a distal end 124 opposite the proximal end 122, and optionally, a shaft electrode 125 located proximate the distal end 124. Additionally, the catheter body 102 includes an inner shaft 126 having a distal end 128. As shown, the proximal end 122 of the outer shaft 120 is attached to and extends distally from the handle 108.

In embodiments, the outer shaft 120 is tubular so as to define an outer shaft lumen (not shown) extending from the proximal end 122 through the distal end 124 such that the distal end 124 is open. Additionally, the inner shaft 126 is slidably disposed within the outer shaft and is dimensioned such that the inner shaft distal end 128 extends distally beyond the distal end 124 of the outer shaft 120. As further shown, the proximal end 114 of each spline 112 is attached to the distal end 124 of the outer shaft 120, and the distal end 116 of each spline 112 is attached to the distal end 128 of the inner shaft 126. Accordingly, the spline assembly 105 is configured to be transitioned between a radially collapsed configuration, such as shown in FIG. 1B, whereby the spline assembly 105 is dimensioned for slidable insertion through a delivery sheath (not shown), and a radially expanded configuration, as illustrated and explained further elsewhere. Additionally, as will be explained in greater detail elsewhere herein, the inner shaft 126 is coupled to the actuator 110, such that the user can change the relative axial position of the distal ends 128, 124 of the inner shaft 126 and the outer shaft 120, respectively, via manipulation of the actuator 110. In this way, the aforementioned transition of the spline assembly 105 configuration is accomplished through axial extension and retraction of the inner shaft 124 relative to the outer shaft 120 using the actuator 110.

FIGS. 1C-1D are plan views of the distal end portion of the ablation catheter 100 showing the spline assembly 105 in different exemplary states of deployment. In the configuration illustrated in FIG. 1C, the distal end 128 of the inner shaft 126 is fully retracted relative to the distal end 124 of the outer shaft 120, such that the spline assembly 105 assumes a minimum axial length. In this configuration, the splines 112 each form a petal-shaped configuration. In embodiments, the spline assembly 105 can assume a form factor similar to that illustrated in FIGS. 26A-26E, 28A-28E and 29A-29B, and described in the corresponding detailed description, of commonly-assigned U.S. Pat. No. 10,172,673, which is incorporated herein by reference in its entirety for all purposes. The fully expanded configuration illustrated in FIG. 1C is particularly suited for spanning a target pulmonary vein from within the left atrium for performing a pulmonary vein isolation via pulsed field ablation.

FIG. 1D illustrates the spline assembly 105 in a partially expanded configuration. In this configuration, the axial position of the distal end 128 of the inner shaft 126 is intermediate that of the fully extended position shown in FIG. 1B, and the fully retracted position shown in FIG. 1C. The geometry of the spline assembly 105 illustrated in FIG. 1D may, in some respects, be similar to conventional splined basket catheter arrangements as are known in the relevant arts.

Retraction and extension of the distal end 128 of the inner shaft 126 relative to the outer shaft 120 is controlled by the user through axial displacement (either proximally or distally) of the actuator 110.

In the illustrated embodiment, the inner shaft 126 includes a lumen so as to be configured to receive and be deployed over a guide wire 130, although in embodiments this feature is omitted.

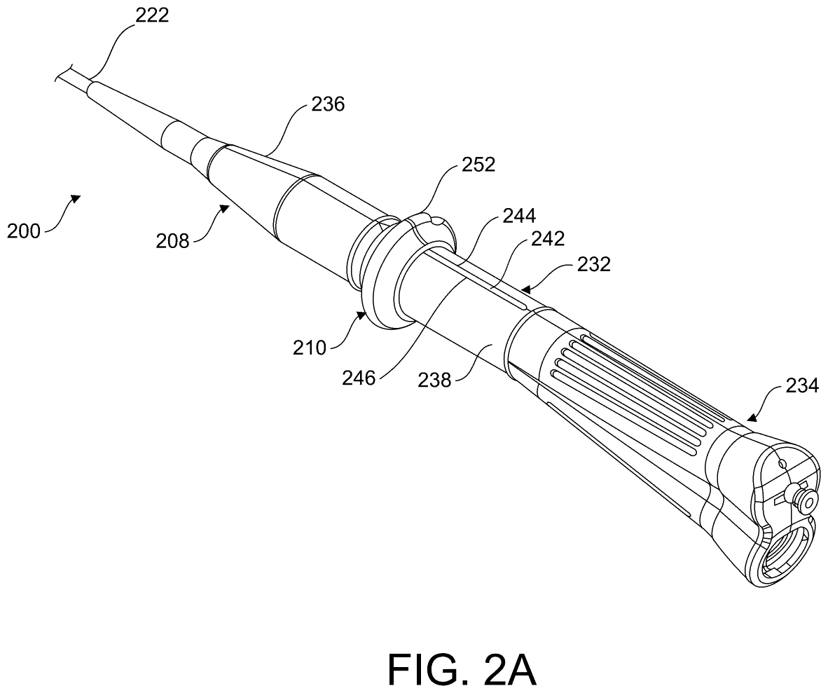

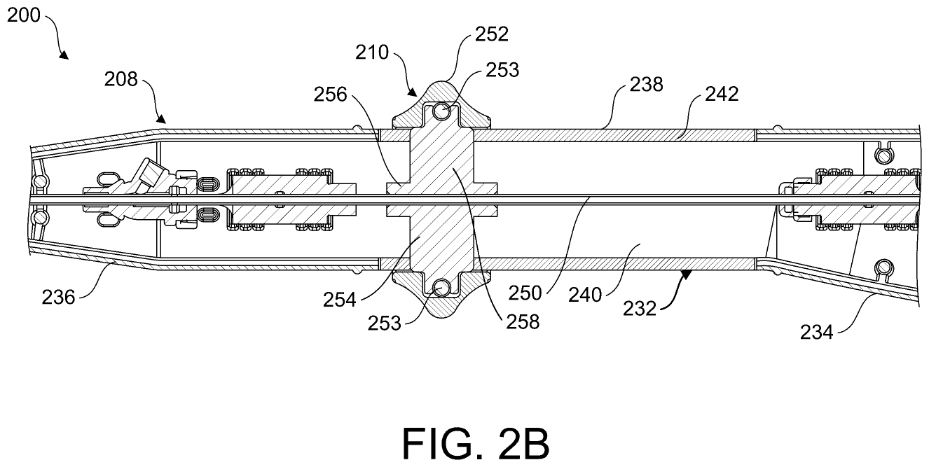

FIGS. 2A and 2B are partial isometric and cross-sectional views, respectively, of an ablation catheter 200 according to embodiments of the present disclosure. The ablation catheter 200 is identical to the catheter 100 and includes a handle 208 and an actuator assembly 210 for changing the configuration, e.g., geometry, of an expandable spline assembly (e.g., the spline assembly 105 of the catheter 100).

Referring to FIGS. 2A and 2B collectively, as shown, the handle 208 includes a handle body 232 having a body proximal end 234, a body distal end 236, an outer surface 238 extending from the body proximal end 234 to the body distal end 236. The handle body 232 is in the form of a shell defining an interior chamber 242. As further shown, the handle body 232 includes an axial slot 242 bounded by opposed lateral edges 244, 246. Additionally, a proximal portion 250 of the inner shaft extends through the handle body 232 within the interior chamber 242. As further shown, a proximal end 222 of the outer shaft is attached to and extends from the body proximal end 234.

The skilled artisan will recognize that FIGS. 2A and 2B illustrate additional features, e.g., flushing components, electrical and electronic components, and the like that are not critical to the present disclosure and are thus not described in detail.

In embodiments, the actuator 210 is slidably coupled to the handle body 232 between the body proximal end 234 and the body distal end 236. As shown, the actuator 210 includes a slider knob 252 disposed about and fully circumscribing the outer surface 238 of the handle body 232. In the various embodiments, the slider knob 252 is configured to be axially displaceable by a user, i.e., in the proximal-distal direction of the handle body 232.

In the illustrated embodiment, the slider knob 252 comprises two pieces, e.g., half-shells, to facilitate assembly, which are secured together by fasteners 253, e.g., screws. In other embodiments, the portions of the slider knob 252 may be secured together by other means, e.g., snap-fit connections, adhesive, and the like.

As further shown, the actuator 210 additionally includes an inner actuator member 254 attached to and extending from the slider knob 252 into the interior chamber 240 of the handle 208. In the illustrated embodiment, the inner actuator member 254 includes a hub 256 and a radial extension 258. As shown, the hub 256 is disposed about and fixedly secured to the proximal portion 250 of the inner shaft, and the radial extension 258 extends from the hub 256 through the axial slot 242 to the slider knob 252. As such, the slider knob 252 is operatively coupled to the proximal portion 250 of the inner shaft such that axial displacement of the slider knob 252 by the user causes the inner shaft to translate axially relative to the outer shaft 222, which in turn changes the configuration of the spline assembly as described in connection with FIGS. 1B-1D. In embodiments, the axial slot 242 has a width that is sized to allow axial movement of the slider knob 252 while constraining rotation of the inner actuator member 254 during axial displacement of the slider knob 252.

Typical clinical procedures utilizing an ablation catheter such as the catheter 200 require the user to manipulate, e.g., rotate the handle 208 to place the spline assembly and corresponding electrodes at the desired location and orientation relative to the target tissue site. In existing ablation catheters having spline assemblies similar to those of the present disclosure, the mechanism for displacing the inner shaft is typically limited to a slider member extending from only a portion of the circumference of the handle. As such, in some instances the aforementioned slider member may not consistently be accessible to the user without difficulty. In contrast, as can be seen particularly in FIG. 2A, the configuration of the actuator 210 of the ablation catheter 200, with the slider knob 252 fully encircling or circumscribing the outer surface 238 of the handle body 232, greatly enhances usability of the ablation catheter 200 by providing the user with free access to the slider knob 252 regardless of the overall orientation of the handle 208.

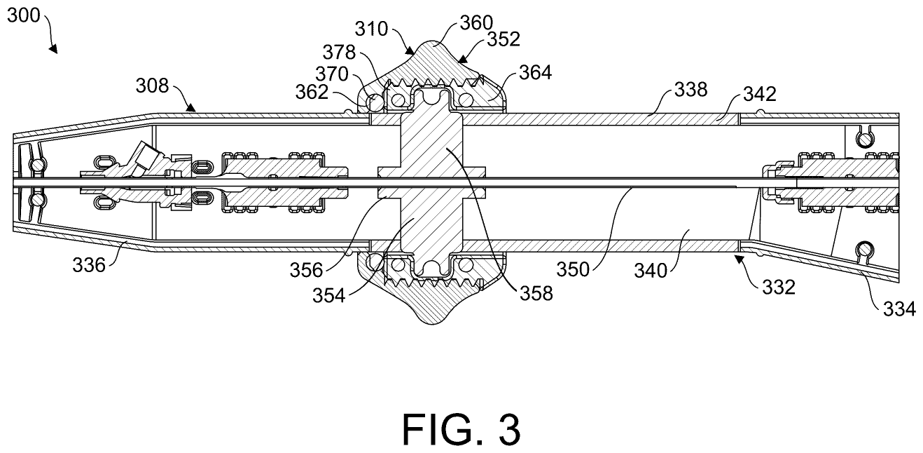

FIG. 3 is a partial cross-sectional view of an ablation catheter 300 according to embodiments of the present disclosure. The ablation catheter 300 is identical to the catheters 100 and 200 except as described and illustrated in connection with FIG. 3. In particular, the ablation catheter 300 has an actuation means for controlling the configuration of the spline assembly that includes an integrated locking arrangement for releasably and selectively locking the axial position of the actuator, and consequently, the configuration of the spline assembly.

As shown, the ablation catheter 300 includes a handle 308 and an actuator assembly 310 for changing the configuration, e.g., geometry, of an expandable spline assembly (e.g., the spline assembly 105 of the catheter 100). The handle 308 includes a handle body 332 having a body proximal end 334, a body distal end 336, an outer surface 338 extending from the body proximal end 334 to the body distal end 336. The handle body 332 is in the form of a shell defining an interior chamber 342. As further shown, the handle body 332 includes an axial slot 342. Additionally, a proximal portion 350 of the inner shaft extends through the handle body 332 within the interior chamber 342.

In embodiments, the actuator 310 is slidably coupled to the handle body 332 between the body proximal end 334 and the body distal end 336. As shown, the actuator 310 includes a slider knob 352 disposed about and fully circumscribing the outer surface 338 of the handle body 332 and configured to be axially displaceable by a user, i.e., in the proximal-distal direction of the handle body 332.

As further shown, the actuator 310 additionally includes an inner actuator member 354 attached to and extending from the slider knob 352 into the interior chamber 340 of the handle 308. In the illustrated embodiment, the inner actuator member 354 includes a hub 356 and a radial extension 358. As shown, the hub 356 is disposed about and fixedly secured to the proximal portion 350 of the inner shaft, and the radial extension 358 extends from the hub 356 through the axial slot 342 to the slider knob 352. As such, the slider knob 352 is operatively coupled to the proximal portion 350 of the inner shaft such that axial displacement of the slider knob 352 by the user causes the inner shaft to translate axially relative to the outer shaft (not shown), which in turn changes the configuration of the spline assembly as described in connection with FIGS. 1B-1D.

As shown in FIG. 3, the slider knob 352 includes an outer knob element 360 having a forward inner surface 362, and an inner knob element 364 having a forward portion 368 in threaded engagement with the outer knob element 360. The outer knob element 360 is rotatable about the handle body 332. The inner knob element 364 is securely attached to the radial extension 358 of the inner actuator member 354 and thus is rotationally fixed relative to the handle body 332. As further shown, a compression element 370, e.g., an O-ring or gasket, is disposed about the handle body 332 and captured between the forward inner surface 362 of the outer knob element 362 and the forward portion 368 of the inner knob element 364.

Accordingly, because the inner knob element 364 cannot rotate relative to the handle body 332, rotation of the outer knob element 360 in a first direction causes the inner knob element 364 to move in a distal direction (due to the threaded engagement between the outer and inner knob elements 360, 364) to compress the compression element 370 between the handle body outer surface 338, the forward inner surface 362 of the outer knob element 360, and the forward portion 368 of the inner knob element 364 to lock the axial position of the slider knob 352. Additionally, rotation of the outer knob element 360 by the user in a second direction opposite the first direction relieves the compression on the compression element 370 and permits the slider knob 352 to be axially displaced. Accordingly, the design of the actuator 310 permits the user to selectively lock and unlock the axial position of the slider knob 352 along the handle body 332.

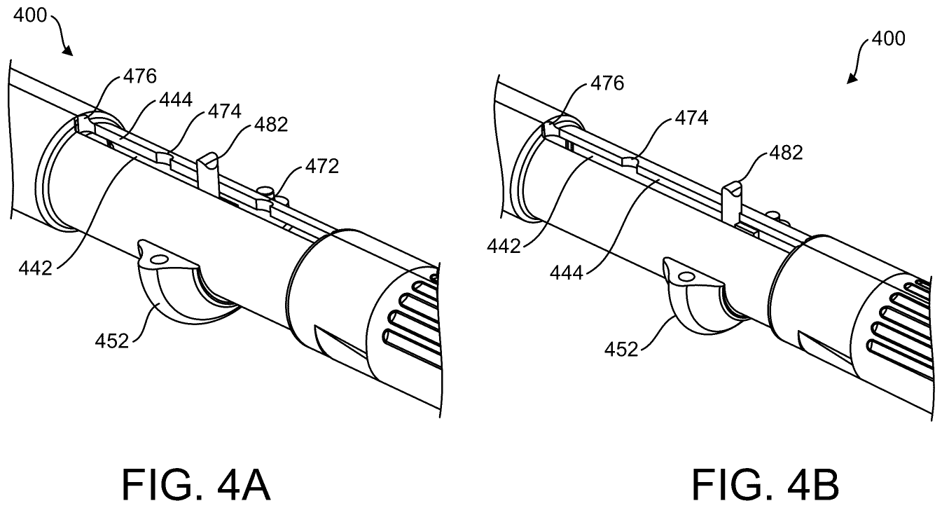

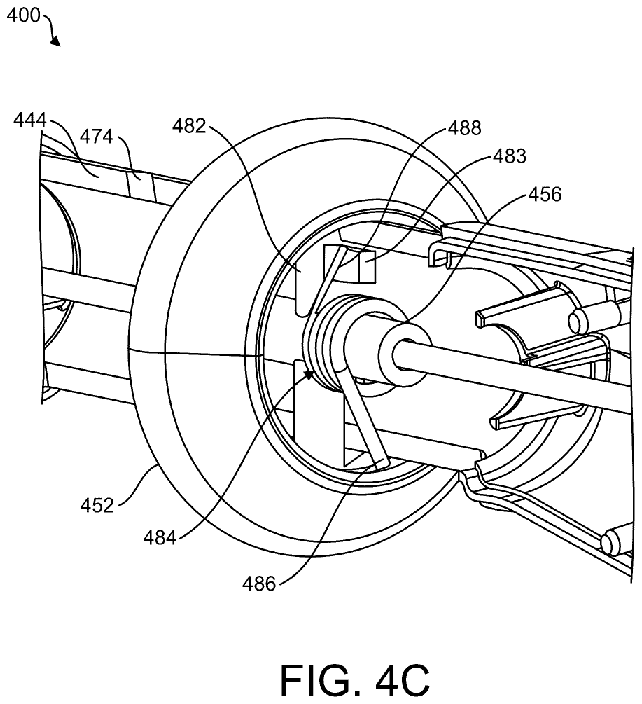

FIGS. 4A-4C are partial isometric views of a portion of an alternative ablation catheter 400 having a handle and actuator featuring a locking arrangement configured to automatically lock the actuator in one or more discrete, pre-defined axial positions. Except as illustrated and described in connection with FIGS. 4A-4C, the catheter 400 can be constructed in the same manner as the aforementioned catheters 100, 200 and 300 described above.

Referring collectively to FIGS. 4A-4C, the handle body of the catheter 400 includes an axial slot 442 bounded by lateral edges, including a lateral edge 444. The catheter 400 further includes an actuator including a slider knob 452 coupled to a hub 456 that is disposed about and secured to the proximal portion of the inner shaft in the same manner as described above in connection with the ablation catheters 100, 200 and 300. Additionally, in the illustrated embodiment, formed in the lateral edge 244 are a plurality of detents 472, 474 and 476. The detents 472, 474 and 476 are spaced from one another along the lateral edge 444 at respective locations corresponding to pre-determined configurations of the spline assembly (see FIGS. 1B-1D). In other embodiments, more or fewer detents may be disposed along the lateral edge 444.

In the illustrated embodiment, the aforementioned locking arrangement includes a locking pin 482 having an axial projection 483, and a torsion spring 484 having a first free end 486 and a second free end 488. In embodiments, the locking pin 482 is attached to the hub 456 and/or the slider knob 452 and extends outward from the hub 456 through the axial slot 442 to a position adjacent to and in contact with the lateral edge 444, and the axial projection 483 extends axially from the locking pin 482 inside the interior chamber of the handle.

Additionally, the torsion spring 484 is disposed about the hub 456. The first free end 486 slidably engages an inner surface of the handle body, while the second free end 488 is positioned to abut and engage the axial projection 483. Accordingly, the torsion spring 484 operates to apply a biasing force to the locking pin 482 and thereby bias the locking pin 482 toward the lateral edge 444, while permitting axial displacement of the slider knob 452, and thus by extension the inner shaft, when the locking pin 482 is located between any of the detents 472, 472, 476. However, the biasing force applied to the locking pin 482 causes the locking pin 482 to automatically engage the detents 472, 472, 476 to releasably lock the axial position of the slider knob 452 at a location corresponding to the respective detent. Additionally, in embodiments, the torsion spring 484 is designed and the axial slot 442 are dimensioned to permit limited rotation of the slider knob 452 sufficient to counteract the biasing force to permit the locking pin 482 to be disengaged from the corresponding detent 472, 472, 476.

As previously described, the respective locations of the detents 472, 472, 476 can be selected so as to automatically and releasably lock the slider knob 452, and thus the inner shaft, in pre-determined locations corresponding to desired pre-determined configurations of the spline assembly. With reference to FIGS. 1B-1D in addition to FIGS. 4A-4C, for example, in embodiments, the detent 472 may be located to automatically lock the slider knob 452 in the axial position corresponding to the fully expanded configuration of the spline assembly 105 illustrated in FIG. 1C. Similarly, the detent 474 may be located to automatically lock the slider knob 452 in the axial position corresponding to the partially expanded configuration of the spline assembly 105 illustrated in FIG. 1D. In the same manner, the detent 476 may be located to automatically lock the slider knob 452 in the axial position corresponding to the fully collapsed configuration of the spline assembly 105 illustrated in FIG. 1B. Additional configurations of the spline assembly 105 can be accommodated by the inclusion of additional detents.

In other embodiments, alternative means may be incorporated to automatically or selectively lock the axial position of the actuator mechanism and the inner shaft and, consequently, the configuration of the spline assembly. By way of example only, in one embodiment, the actuator may include a collet mechanism that clamps to the handle body upon rotation of the slider knob to frictionally inhibit axial displacement of the slider knob, and consequently, the inner shaft. In alternative embodiments, the handle body and the slider knob (or other actuator component) may have geometric shapes that create frictional engagement therebetween upon rotation of the slider knob when the user desires to lock the slider knob in place. For example, in one embodiment, the adjacent surfaces of the slider knob and the handle body may each have elliptical profiles, such that rotation of the slider knob relative to the handle body creates an adjustable degree of interference and friction therebetween.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical catheter comprising:
a catheter body comprising:
a tubular outer shaft having a proximal end and an open distal end opposite the proximal end;
an inner shaft slidably disposed within the outer shaft having a proximal portion and an opposite distal end, the inner shaft being dimensioned such that the inner shaft distal end extends distally beyond the distal end of the outer shaft;
a spline assembly comprising a plurality of splines each having a spline proximal end attached to the distal end of the outer shaft, and an opposite spline distal end attached to the distal end of the inner shaft, the spline assembly configured to be transitioned between a collapsed configuration dimensioned for slidable insertion through a delivery sheath, and an expanded configuration;
a handle comprising a handle body having a body proximal end, a body distal end, an outer surface extending from the body proximal end to the body distal end, the handle body defining an interior chamber, wherein the proximal end of the outer shaft is attached to and extends from the body proximal end, and the proximal portion of the inner shaft is disposed within the interior chamber; and
an actuator slidably coupled to the handle body between the body proximal end and the body distal end, the actuator comprising a slider knob disposed about and fully circumscribing the outer surface of the handle body such that the slider knob can be axially displaced by a user, wherein the slider knob is further operatively coupled to the proximal portion of the inner shaft such that axial displacement of the slider knob by the user causes the inner shaft to translate axially relative to the outer shaft to transition the spline assembly between the collapsed configuration and the expanded configuration.

2. The medical catheter of claim 1, wherein the actuator further comprises an inner actuator member attached to and extending from the slider knob into the interior chamber of the handle, and wherein the inner actuator member is fixedly secured to the proximal portion of the inner shaft within the interior chamber and operatively couples the inner shaft to the slider knob.

3. The medical catheter of claim 2 wherein the inner actuator member includes a hub disposed about and fixedly secured to the proximal portion of the inner shaft, and a radial extension extending from the hub to the slider knob.

4. The medical catheter of claim 3, wherein the handle body includes an axial slot defined by opposed first and second lateral edges, and wherein the radial extension extends through the axial slot and the first and second lateral edges constrain rotation of the inner actuator member during axial displacement of the slider knob.

5. The medical catheter of claim 4, wherein the actuator is configured such that the user can lock the slider knob in an axial position.

6. The medical catheter of claim 5, wherein the slider knob includes an outer knob element that is rotatable about the handle body, wherein rotation of the outer knob element by the user in a first direction locks the axial position of the slider knob, and rotation of the outer knob element in a second direction opposite the first direction permits the slider knob to be axially displaced.

7. The medical catheter of claim 6, wherein the slider knob further comprises an inner knob element that is rotationally fixed relative to the handle body and includes a forward portion disposed radially inward of and in threaded engagement with the outer knob element, wherein rotation of the outer knob element causes the inner knob element to translate axially to selectively lock and unlock the slider knob relative to the handle body.

8. The medical catheter of claim 7, wherein the outer knob element includes a forward inner surface, and wherein the handle includes a compression element disposed about the handle body between the forward inner surface and the forward portion of the inner knob element, wherein rotation of the outer knob element in the first direction causes the inner knob element to move in a distal direction to compress the compression element between the handle body outer surface, the forward inner surface of the outer knob element, and the forward portion of the inner knob element to lock the axial position of the slider knob.

9. The medical catheter of claim 4, wherein the first lateral edge includes a first detent at a first location along the lateral edge, and wherein the locking arrangement comprises a locking pin extending radially from the hub through the axial slot, wherein the locking pin is biased toward the first lateral edge so as to automatically engage the first detent to releasably lock the axial position of the slider knob corresponding to the first location.

10. The medical catheter of claim 9, wherein the locking arrangement further includes a torsion spring disposed about the hub, the torsion spring including a first free end frictionally engaged with an inner surface of the handle body, and an opposite second free end positioned to engage and apply a biasing force to the locking pin to bias the locking pin toward the first lateral edge.

11. The medical catheter of claim 10, wherein the slider knob is configured to be rotatable to counteract the biasing force and selectively disengage the locking pin from the first detent.

12. The medical catheter of claim 11, wherein the first lateral edge includes a second detent at a second location along the first lateral edge, and a third detent at a third location along the first lateral edge, the first, second and third locations being spaced from one another and corresponding to pre-determined configurations of the spline assembly.

13. A medical catheter comprising:
a catheter body comprising a tubular outer shaft and an inner shaft slidably disposed within the outer shaft and dimensioned to extend distally beyond the outer shaft;
a radially expandable and radially collapsible spline assembly comprising a plurality of splines each having a spline proximal end attached to the distal end of the outer shaft, and an opposite spline distal end attached to the distal end of the inner shaft;
a handle coupled to the catheter body, the handle having a handle proximal end and a handle distal end, wherein a proximal end of the outer shaft is attached to and extends from the handle proximal end, and a proximal portion of the inner shaft is slidably disposed within the handle; and
an actuator comprising a slider knob slidably disposed about and fully circumscribing an outer surface of the handle, wherein the slider knob is operatively coupled to the proximal portion of the inner shaft such that axial displacement of the slider knob by a user causes the inner shaft to translate axially relative to the outer shaft to selectively control an amount of expansion of the spline assembly.

14. The medical catheter of claim 13, wherein the actuator is configured such that the user can lock the slider knob in an axial position.

15. The medical catheter of claim 13, wherein the actuator further comprises a locking arrangement configured to releasably lock the actuator in one or more pre-defined axial positions.

16. A handle assembly for a medical catheter having a catheter body including a tubular outer shaft, an inner shaft slidably disposed within the outer shaft, and a radially expandable and collapsible splined electrode assembly disposed at a distal end of the catheter body, the handle assembly comprising:
a handle comprising a handle body having a body proximal end, a body distal end, an outer surface extending from the body proximal end to the body distal end, the handle body defining an interior chamber configured to slidably receive a proximal portion of the inner shaft; and
an actuator slidably coupled to the handle body between the body proximal end and the body distal end, the actuator comprising a slider knob disposed about and fully circumscribing the outer surface of the handle body such that the slider knob can be axially displaced by a user, wherein the slider knob is further configured to be operatively coupled to the proximal portion of the inner shaft such that axial displacement of the slider knob by the user can cause the inner shaft to translate axially relative to the outer shaft to transition the spline assembly between a collapsed configuration and an expanded configuration.

17. The handle assembly of claim 16, wherein the handle body includes an axial slot defined by opposed first and second lateral edges, and the actuator further comprises an inner actuator member including a hub configured to be disposed about and fixedly secured to the proximal portion of the inner shaft, and a radial extension extending from the hub through the axial slot to the slider knob.

18. The handle assembly of claim 17, wherein the slider knob includes an outer knob element that is rotatable about the handle body, an inner knob element that is rotationally fixed relative to the handle body and includes a forward portion disposed radially inward of and in threaded engagement with the outer knob element, wherein rotation of the outer knob element causes the inner knob element to translate axially to selectively lock and unlock the slider knob relative to the handle body.

19. The handle assembly of claim 18, wherein the outer knob element includes a forward inner surface, and wherein the handle further includes a compression element disposed about the handle body between the forward inner surface and the forward portion of the inner knob element, wherein rotation of the outer knob element in a first direction causes the inner knob element to move in a distal direction to compress the compression element between the handle body outer surface, the forward inner surface of the outer knob element, and the forward portion of the inner knob element to lock the axial position of the slider knob.

20. The handle assembly of claim 17, wherein the first lateral edge includes a plurality of axially-spaced detents, and wherein the actuator includes a locking arrangement comprises a locking pin extending radially from the hub through the axial slot, wherein the locking pin is biased toward the first lateral edge so as to automatically engage the one of the plurality of detents to releasably lock the axial position of the slider knob corresponding to a predetermined configuration of the spline assembly.
